(19) United States
(12) Patent Application Publication
(10) Pub. No.: US 2010/0015697 A1
(43) Pub. Date: Jan. 21, 2010

(54) MICRO-FLUIDIC CELL MANIPULATION AND HOLDING DEVICE

(76) Inventors: Michael Carl Junger, Brookfield (AU); Katie Lou Paten, Marsden (AU); Kim John Giliam, Wakerley (AU); Jeremy Thompson, Grange (AU); David Sean O'Brien, Redland Bay (AU)

Correspondence Address: BRINKS HOFER GILSON & LIONE/CHICAGO/COOK, PO BOX 10395, CHICAGO, IL, 60610, US

(21) Appl. No.: 12/501,043

(22) Filed: Jul. 10, 2009

Related U.S. Application Data
(60) Provisional application No. 61/135,049, filed on Jul. 16, 2008.

(51) Int. Cl. C12M 1/02

(52) U.S. Cl. 435/307.1

(57) ABSTRACT

A micro-fluidic manipulation device is formed using a plastic material or glass. There is at least one well in the device. The well has a base and a side wall and is dimensioned to receive a cell. There is at least one base fluid port in the base of the well and side fluid ports in the side wall. Each of the base fluid port and the side fluid ports are of a cross sectional area to prevent a cell passing therethrough. Selected flow of fluid through the base port in use assists in supporting a cell in the well and selected flow of fluid through the base port and the side ports in use assists in rotating the cell in the well. The well can have a retention section and a loading section, the loading section being above and larger than the retention section. The loading section can have a loading channel extending to it or alternatively an access hatch. The side fluid ports open into a lower region of the loading section.

### RELATED APPLICATIONS

The present patent document claims the benefit of the filing date under 35 U.S.C. §119(e) of Provisional U.S. patent application Ser. No. 61/135,049, filed Jul. 16, 2008, which is hereby incorporated by reference.

### BACKGROUND

1. Technical Field

The present invention relates to a device for manipulating or holding cells, such as culturing cells or tissues. More specifically, the invention relates to a micro-fluidic device for manipulating or holding cells or tissues.

2. Background of the Invention

Micro-fluidics is the technology used to design, model, manufacture and mass-produce micro-systems that handle fluids—gases, vapours or liquids in volumes that can be as small as micro, nano or pico liters. Active and passive microstructures control the flow and mixing of the fluids to produce physical, chemical, biochemical and microbiological reactions in a rapid, cost-effective manner. Micro-fluidics have a range of applications, including the handling and culture of cells and the automation of highly manual laboratory processes, such as in vitro fertilization (IVF), onto a single substrate.

When handling an embryonic cell, it is desirable to be able to hold it at a certain orientation or to manipulate it into a selected orientation for morphological examination and then hold it at that orientation for observation and treatment. This is particularly the case during intracytoplasmic sperm injection (ICSI), where the orientation of the polar body of the oocyte to the injection site is very important.

### SUMMARY

The invention will be discussed in relation to its application for cell manipulation and treatment but its application is not so limited and can also relate to cell and tissue culture more generally. The invention has particular application to mammalian cell culture including mammalian cell replication and/or reproduction. In one application, the invention is used for culturing embryos for IVF.

The invention will be discussed in relation to embryonic cells but is not so limited and may apply to other forms of cells or similar organisms.

The present invention seeks to provide a device which enables manipulation and treatment of a cell at a micro-fluidic volume or level or to at least provide the practitioner in the field with a useful device.

In one embodiment, the invention relates to a micro-fluidic manipulation device. The device includes a body, at least one well in the formed in the body, the well including a base and a side wall, the well being dimensioned to receive a cell selected from the group consisting of an oocyte, embryonic cell or somatic cell. The device also includes at least one base fluid port in the base of the well and at least one side fluid port in the side wall, each of the at least one base fluid port and the at least one side fluid port including a cross sectional area dimensioned to prevent the cell passing therethrough. Selected flow of fluid through the base fluid port in use assists in holding the cell; and selected flow of fluid through the base fluid port and the at least one side port in use assists in rotating the cell in the well.

Preferably, the device includes two pairs of diametrically opposed side fluid ports in the side wall.

Preferably, each of the at least one base fluid port and the at least one side fluid port comprises a non-circular cross sectional area, whereby to prevent a seal being formed when the cell is retained thereon by the selected fluid flow. The non-circular cross sectional area can be selected from shapes, such as star shape, elliptical shape, hemispherical, hemi-elliptical, square shape, rectangular shape, and other suitable shape.

The base fluid port may be used to supply to or withdraw fluid from the well. For instance, it would be possible to draw fluid from the base port in order to hold the oocyte, embryonic cell or somatic cell while exchanging cell media through the port.

In another embodiment, the well includes a retention section and a loading section, the loading section being above and larger than the retention section, and the at least one side fluid port opening into a lower region of the loading section. The loading section may include a loading channel extending thereto. Alternatively, the loading region may be accessed from directly over the top of the loading port. Each of the at least one base fluid port and the at least one side fluid port may be supplied by micro-fluidic flow ducts in the body.

There can be further included at least one cell media withdrawal duct extending from the well. By having more than one separate cell media exchange duct, various media may be switched between/flushed. The media withdrawal duct or ducts extending from the well can comprise a cross sectional area dimensioned to prevent the cell passing there through.

Alternatively, there can be a plurality of cell media withdrawal ducts extending from the well, where each media withdrawal duct comprises a cross sectional area dimensioned to prevent the cell passing therethrough.

The media withdrawal duct may be at an upper end of the loading cell whereby to provide minimal disturbance to flow of fluid in the well.

Accordingly, the invention relates to a micro-fluidic device which can hold a cell, such as an oocyte embryonic cell, or a somatic cell, and provides fluid flow through respective ports so that the cell can be manipulated, such as being rotated to a selected position or the cell can be held in place for any necessary treatment.

### BRIEF DESCRIPTION OF THE DRAWINGS

This then generally describes the invention but to assist with understanding reference will now be made to the accompanying drawings, which show certain selected embodiments of the invention.

In the drawings:

FIG. 1 shows a plan view of a first embodiment of cell manipulation and holding device according to the present invention;

FIG. 2 shows a part cross sectional view of the embodiment of cell manipulation and holding device along the line 2-2′ in FIG. 1;

FIG. 3 shows a part cross sectional view of the embodiment of cell manipulation and holding device along the line 3-3′ in FIG. 1;

FIG. 4 shows a part cross sectional view of the embodiment of cell manipulation and holding device along the line 4-4′ in FIG. 1;

FIG. 5 shows a detailed view of one embodiment of the well of a cell manipulation and holding device according to the present invention;

FIG. 6 shows a detailed view of an alternative embodiment of the well of a cell manipulation and holding device according to the present invention;

FIGS. 7A to 7D show still further embodiments of the well of a cell manipulation and holding device according to the present invention;

FIG. 8 shows an alternative embodiment of cell manipulation and holding device according to the present invention;

FIG. 9 shows detail of an alternative embodiment of well particularly showing an arrangement of diffuse outlet ports;

FIG. 10 shows an alternative embodiment of cell manipulation and holding device according to the present invention; and

FIG. 11 shows detail of the embodiment of FIG. 10 in cross section along the line 11-11′ in FIG. 10.

### DETAILED DESCRIPTION

The following detailed description and appended drawings describe and illustrate various exemplary embodiments of the invention. The description and drawings serve to enable one skilled in the art to make and use the invention.

Definitions:

It should be understood that the terms “a” and “an” as used above and elsewhere herein refer to “one or more” of the enumerated components. For example, “a” cell refers to one cell or a mixture comprising two or more cells.

As used herein the terms “comprise(s),” “include(s),” “having,” “has,” “contain(s),” and variants thereof, are intended to be open-ended transitional phrases, terms, or words that do not preclude the possibility of additional acts or structure.

The term “oocyte” refers to a cell from which an egg or ovum develops by meiosis; a female gametocyte.

The terms “embryonic cell,” “embryonic stem cell,” or “pluripotent stem cell,” may be used interchangeably and refer to one of the cells that are self-replicating, are derived from embryos, such as human embryos or human fetal tissue, and are known to develop into cells and tissues of the three primary germ layers. Although human pluripotent stem cells may be derived from embryos or fetal tissue, such stem cells are not themselves embryos. (See the National Institutes of Health Guidelines for Research Using Human Pluripotent Stem Cells.) “Self-replicating” means the cell can divide and form cells indistinguishable from it. The “three primary germ layers”—called the ectoderm, mesoderm, and endoderm—are the primary layers of cells in the embryo from which all tissues and organs develop.

The term “somatic cell” refers to any cell forming the body of an organism, as opposed to germline cells. Somatic cells may be of mammalian and non-mammalian origin. For example, in mammals, germline cells (also known as “gametes”) are the spermatozoa and ova which fuse during fertilization to produce a cell called a zygote, from which the entire mammalian embryo develops. Every other cell type in the mammalian body—apart from the sperm and ova, the cells from which they are made (gametocytes) and undifferentiated stem cells—is a somatic cell: internal organs, skin, bones, blood, and connective tissue are all made up of somatic cells.

The present invention relates to a cell manipulation and holding device and methods of use and manufacture thereof. The cells to be manipulated or held include but are not limited to an oocyte, embryonic cell or somatic cell.

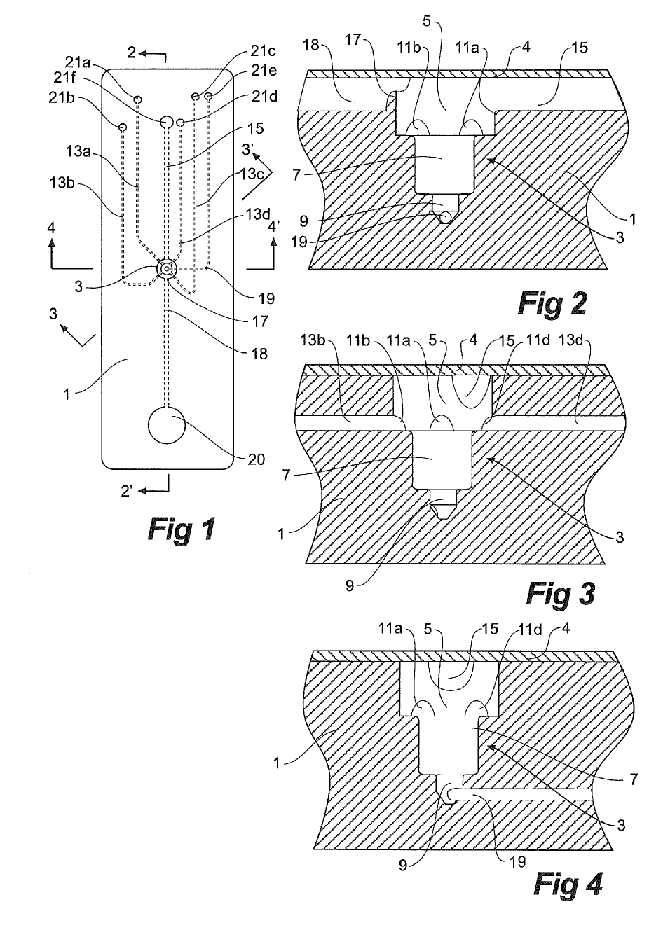

Referring to FIGS. 1 to 4, in one embodiment, a cell manipulation and holding device 1 according to the present invention has a substantially rectangular body. The body can be made from glass or plastics material and may be assembled from a plurality of layers to facilitate formation of the features as discussed below. The body may have coatings or the like as is well known in the art. The body 1 has a cover plate 4, which can be removed to allow access to the well.

The device 1 has a well 3, which as can be seen in FIG. 2. The well has a loading section 5 and a retention section 7 and a base fluid port 9. The retention section 7 is below the loading section 5 and the base fluid port 9 is below the retention section 7. A number of control ports 11a to 11d extend into the loading section towards the lower end of the loading section. The control ports are spaced evenly around the loading section. In this embodiment, the control ports are arranged in opposite pairs, but in an alternative embodiment there may be three ports at 120 degrees to each other or any other convenient number. The control ports are of a lesser diameter than the expected diameter of a cell to be handled in the well, such an oocyte, embryonic cell, or somatic cell, to prevent a cell from entering the control port.

A series of supply ducts 13a to 13d extends to each control port 11a to 11d, respectively. A loading duct 15 extends to the loading section and a diffuse outlet port 17 and duct 18 extends away from the loading port. The diffuse outlet port 17 is of lesser diameter than the loading port to prevent a cell from moving from the loading chamber into the diffuse outlet port 17. There may be more than one diffuse outlet port so that flow of media out of the well via the diffuse outlet port or ports 17 does not affect significantly the position of a cell in the well. A media withdrawal duct 18 extends to a waste well 20 from which the excess media can be withdrawn.

A manipulation duct 19 extends to the base fluid port 9. Each of the supply ducts 13a to 13d, the loading duct 15, and the manipulation duct 19 extend to suitable connection points 21a to 21f for selective fluid supply to the device. The connection point 21f is larger than the other ports to allow for placement of a cell for transport along the loading duct 15 to the loading section 5 of the well 3.

In use, a cell is placed into the connection point 21 and carried on a fluid flow along the loading duct 15 to the loading section 5 of the well 3. The cell is retained in the retention section 7 for observation and the like. It is desirable to be able to rotate the cell for observation of the cell and hence a low fluid flow is provided through the manipulation duct 19 to lift the cell slightly into the loading section. The flow through one or more of the control ports 11a to 11d is used to rotate the cell as desired. Once a cell has been positioned at a selected orientation, the flow through the manipulation duct 19 is stopped and the cell is allowed to move into the retention section under a low positive pressure from the control ports. At this stage operations can be performed on the cell, such as injection if a suitable aperture (not shown) is provided in the cover plate 4 or the cover plate 4 can be removed to provide access to the well.

Alternatively, once a cell has been positioned at a selected orientation the flow through the manipulation duct 19 can be stopped and fluid withdrawn through the manipulation duct 19 so that the cell is caused to move back into the retention section and held against the base fluid port 9 by suction through the manipulation duct 19.

To prevent a cell held against the base fluid port, either by flow at low positive pressure from the control ports or by withdrawal of fluid from the base fluid port, from causing the cell to jam into and block the base fluid port 9 the base fluid port 9 may be formed in a shape other than circular.

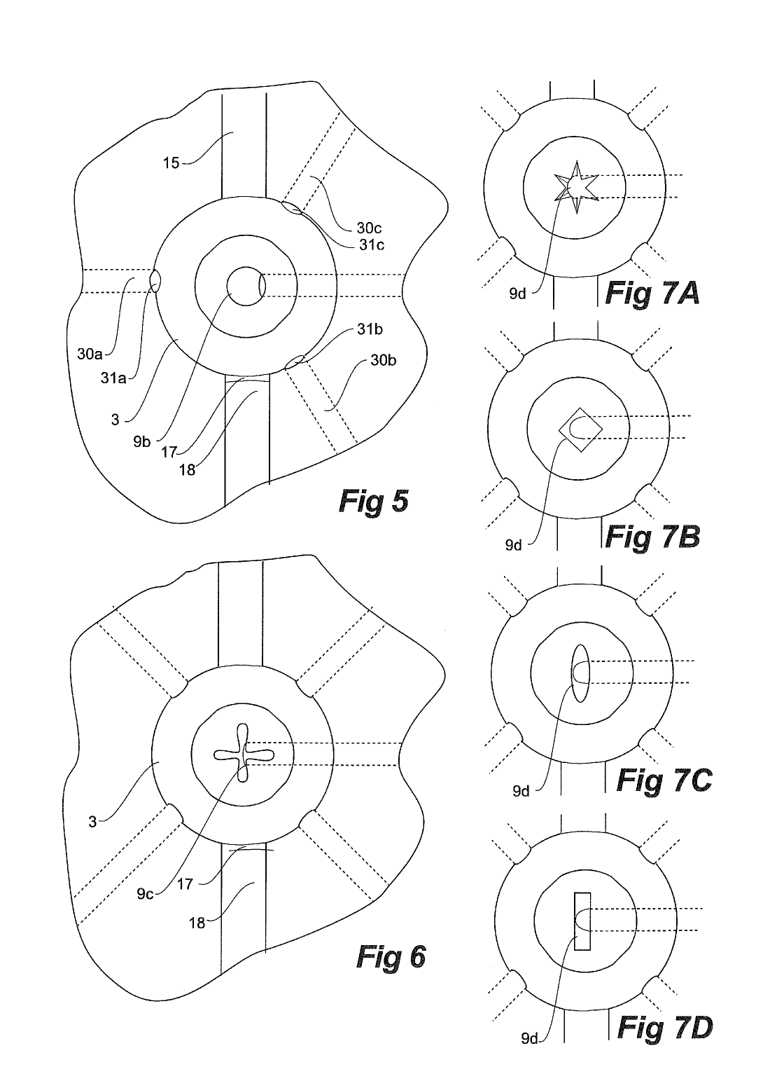

FIG. 5 shows detail of one embodiment of the well 3 in plan view with the base fluid port 9b being circular. Also, in this embodiment there are three supply ducts 30a to 30c and three control ports 11a to 11c.

FIG. 6 shows details of another embodiment of the well 3 in plan view with the base fluid port 9c being cloverleaf shaped.

FIGS. 7A to 7D show further alternative shapes of base fluid ports. In FIG. 7A the base fluid port 9d is of a star shape. In FIG. 7B the base fluid port 9e is of a diamond shape. In FIG. 7C the base fluid port 9f is of an elliptical shape. In FIG. 7A the base fluid port 9g is of a rectangular shape.

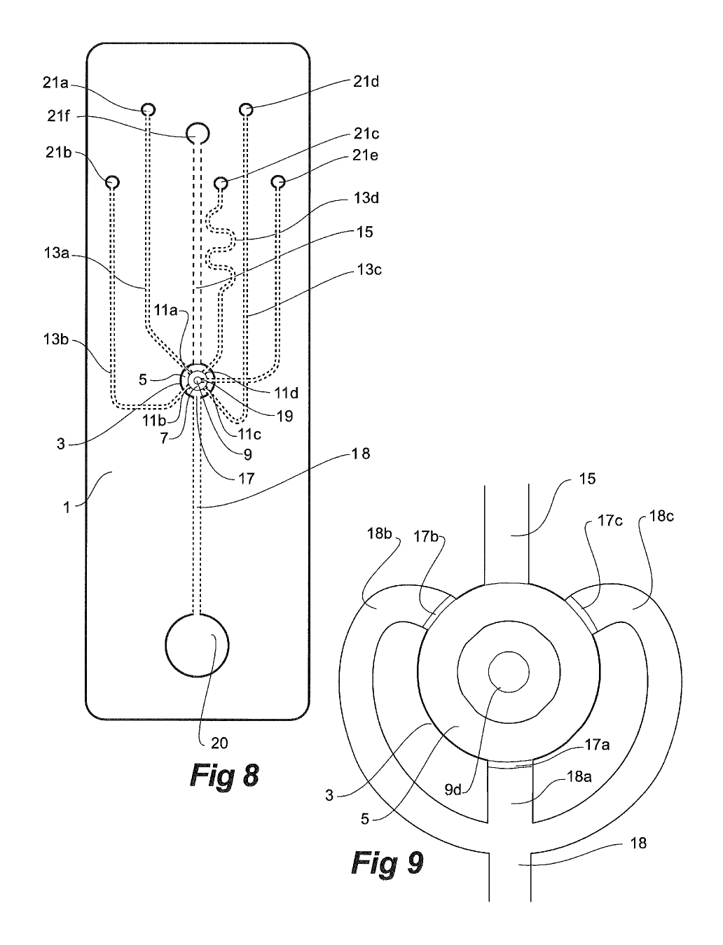

FIG. 8 shows an alternative embodiment of the cell manipulation and holding device according to the present invention. In this embodiment, the same reference numerals have been used for corresponding items as those shown in FIGS. 1 to 4.

In FIG. 8 the device 1 has a well 3. A number of control ports 11a to 11d extend into a loading section 5 towards the lower end of the loading section 5. The control ports are spaced evenly around the loading section. The control ports are of a lesser diameter than the expected diameter of a cell to be handled in the well to prevent a cell from entering a control port. A series of supply ducts 13a to 13d extends to each control port 11a to 11d, respectively. A loading duct 15 extends to the loading section 5 and a diffused outlet duct 18 extends away from a diffused outlet port 17 on the loading section. The diffused outlet port 17 is of lesser diameter than the diameter of the loading port to prevent a cell from moving from the loading chamber into the diffused outlet port 17. There may be more than one diffused outlet port so that flow of media out of the well via the diffused outlet port or ports 17 does not affect significantly the position of a cell in the well. The media withdrawal duct 18 extends to a waste well 20 from which excess media can be withdrawn.

A manipulation duct 19 extends to the base fluid port 9. Each of the supply ducts 13a to 13d, the loading duct 15, and the manipulation duct 19 extend from suitable connection points 21a to 21f for fluid supply to the device. The connection point 21f is larger than the other ports to allow for placement of a cell for transport along the loading duct 15 to the loading section 5 of the well 3.

It is advantageous to have all of the control ducts to be the same length from the respective supply ports 21a to 21d to the control ports 11a to 11d. This ensures that an equal amount of flow in one of these ducts caused the same expected action in the well. Hence the duct 13c is convoluted to make it the same overall length as the others. The respective supply ports 21a to 21d are spaced apart on the body 1 to allow for convenient connection to a suitable fluid supply for each supply port.

FIG. 9 shows details of an alternative embodiment of well particularly showing an arrangement of diffuse outlet ports. In this embodiment, the well 3 has a loading duct 15 and three diffuse outlet ports 17a to 17c with ducts 18a extending to the waste duct 18.

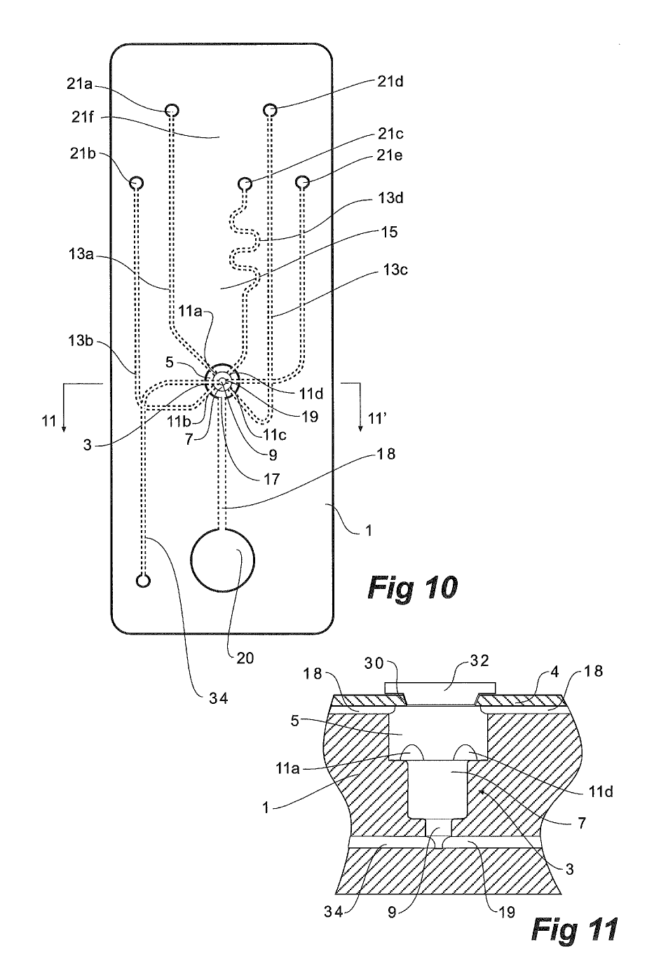

FIG. 10 shows an alternative embodiment of the cell manipulation and holding device according to the present invention. FIG. 11 shows details of the embodiment of FIG. 10 in cross section along the line 11-11′ in FIG. 10. In this embodiment, the same reference numerals have been used for corresponding items as those shown in FIGS. 1 to 4.

In this embodiment, the cell manipulation and holding device according to this embodiment has a substantially rectangular body 1. The body is illustrated as rectangular but can be any other convenient shape. The body can be made from glass or plastics material and may be assembled from a plurality of layers to facilitate formation of the features as discussed herein. The body may have coatings or the like as is well known in the art. The body 1 can have a cover plate 4, which can be removed to allow access to the well.

The device 1 has a well 3, which as can be seen in FIG. 11, has a loading section 5 and a retention section 7 and a base fluid port 9. The retention section 7 is below the loading section 5 and the base fluid port 9 is below the retention section 7. A number of control ports 11a to 11d extend into the loading section towards the lower end of the loading section. The control ports are spaced evenly around the loading section. In this embodiment, the control ports are arranged in opposite pairs, but in an alternative embodiment there may be three ports at 120 degrees to each other, or any other convenient number. The control ports are of a lesser diameter than the expected diameter of a cell to be handled in the well to prevent a cell from entering a control port.

A series of supply ducts 13a to 13d extends to each control port 11a to 11d, respectively. A loading aperture 30 is directly above the loading section, and a diffuse outlet port 17 and duct 18 extend away from the loading port. A removable lid 32 in the loading aperture 30 can be removed to allow access to the well. The diffuse outlet port 17 is of lesser diameter than the expected diameter of a cell which may be placed into the well to prevent a cell from moving from the loading chamber into the diffuse outlet port 17. There may be more than one diffuse outlet port so that flow of media out of the well via the diffuse outlet port or ports 17 does not affect significantly the position of a cell in the well. A media withdrawal duct 18 extends to a waste well 20 from which excess media can be withdrawn.

The base fluid port 9 has two ducts extending to it. A first duct is a supply duct 19 and a second is a withdrawal duct 34. As described above with reference to other embodiments, the base port 9 preferably has a non-circular cross section to enable a cell to be held against the port but still enabling a small flow of media to be drawn into the base fluid port and out of the withdrawal duct 34. This arrangement may allow for a cell be held in a selected orientation for treatment, as discussed above.

Throughout this specification various indications have been given as to the scope of the invention but the invention is not limited to any one of these but may reside in two or more combined together. The examples and embodiments are given for illustration and not for limitation.

In addition to the embodiments described above, the invention includes combinations of the preferred embodiments discussed above, and variations of all embodiments.

1. A micro-fluidic manipulation device comprising
a body;
at least one well in the body, the well comprising a base and a side wall, the well being dimensioned to receive a cell selected from the group consisting of an oocyte, embryonic cell or somatic cell;
at least one base fluid port in the base of the well; and
at least one side fluid port in the side wall,
wherein each of the at least one base fluid port and the at least one side fluid port comprising a cross sectional area dimensioned to prevent the cell passing therethrough,
wherein selected flow of fluid through the base fluid port in use assists in holding the cell, and
wherein selected flow of fluid through the base fluid port and the at least one side fluid port in use assists in rotating the cell in the well.

2. The micro-fluidic manipulation device of claim 1, comprising two pairs of diametrically opposed side fluid ports in the side wall.

3. The micro-fluidic manipulation device of claim 1, wherein each of the at least one base fluid port and the at least one side fluid port comprises a non-circular cross sectional area to prevent a seal being formed when a cell is retained thereon by the selected fluid flow.

4. The micro-fluidic manipulation device of claim 3, wherein the non-circular cross sectional area is of star shape, elliptical shape, hemi-spherical, hemi-elliptical, square shape, or rectangular shape.

5. The micro-fluidic manipulation device of claim 3, wherein the non-circular cross sectional area comprises a filter membrane.

6. The micro-fluidic manipulation device of claim 1, wherein the well comprises
a retention section; and
a loading section, the loading section being disposed above and larger than the retention section, the loading section comprising a loading channel extending thereto and the at least one side fluid port opening into a lower region of the loading section.

7. The micro-fluidic manipulation device of claim 1, wherein each of the at least one base fluid port and the at least one side fluid port are supplied by micro-fluidic flow ducts in the body.

8. The micro-fluidic manipulation device of claim 1, further comprising at least one media withdrawal duct extending from the well.

9. The micro-fluidic manipulation device of claim 8, wherein the media withdrawal duct comprises a cross sectional area dimensioned to prevent a cell passing into the duct.

10. The micro-fluidic manipulation device of claim 1, further comprising a plurality of media withdrawal ducts extending from the well, wherein each media withdrawal duct comprises a cross sectional area dimensioned to prevent a cell passing therethrough.
